(19) United States
(12) Patent Application Publication
(10) Pub. No.: US 2011/0200624 A1
(43) Pub. Date: Aug. 18, 2011

(54) NOVEL PEPTIDES FOR USE IN TRANSFECTION

(75) Inventor: Joel A. Jessee, Mount Airy, MD (US)

(73) Assignee: MOLECULAR TRANSFER, INC., Gaithersburg, MD (US)

(21) Appl. No.: 13/056,930

(22) PCT Filed: Jul. 31, 2009
(86) PCT No.: PCT/US09/52425

§ 371 Date: May 3, 2011

Related U.S. Application Data
(60) Provisional application No. 61/085,253, filed on Jul. 31, 2008.

(51) Int. Cl. A61K 39/395; C07K 2/00; C07K 14/00; C12N 15/63; C07K 9/00; C12N 5/07; C12N 15/87; A61P 35/00

(52) U.S. Cl. 424/178.1; 530/322; 530/409; 435/320.1; 435/325; 435/455

(57) ABSTRACT

Novel peptides derived from antibody complementarity determining regions (CDRs) that enhance delivery of macromolecules into cells, particularly when used in combination with cationic lipids, are provided. The peptides can be combined with cationic lipids, and compositions of cationic lipids associated with enhancer elements, to provide reagents that can complex with macromolecules such as nucleic acids, proteins and peptides and permit introduction of these macromolecules into a variety of cells and tissues in vitro or in vivo with greatly enhanced efficiency compared to other lipid-based reagents. Methods for delivering macromolecules into target cells and tissues using the lipids and enhancer elements are provided.

### BACKGROUND

Methods for using cationic lipids for introduction of macromolecules, such as DNA, RNA, and proteins, into living cells were first described by Feigner et al. See Nature 337:387-388 (1989); Proc. Natl. Acad. Sci. USA 84:7413 (1987). Several cationic lipids have been described in the literature and some of these are commercially available. DOTMA (N[1-(2,3-dioleyloxy)propyl]-N,N,N-trimethylammonium chloride) was the first cationic lipid to be synthesized for the purpose of nucleic acid transfection. See Feigner et al. (Proc. Nat'l Acad. Sci. 84, 7413 (1987); U.S. Pat. No. 4,897,355). DOTMA can be formulated alone or can be combined with DOPE (dioleoylphosphatidylethanolamine) into a liposome, and such liposomes can be used to deliver plasmids into some cells. Other classes of lipids subsequently have been synthesized by various groups. For example, DOGS (5-carboxyspermylglycine-dioctadecylamide) was the first polycationic lipid to be prepared (Behr et al. Proc. Nat'l Acad. Sci. 86,6982 (1989); U.S. Pat. No. 5,171,678) and other polycationic lipids have since been prepared. The lipid DOSPA (2,3-dioleyloxy-N-[2(spermine-carboxamido)ethyl]-N,N-dimethyl-1-propanaminium) has been described as an effective delivery agent (U.S. Pat. No.5,334,761).

In other examples, cholesterol-based cationic lipids, such as DC-Chol (N,N-dimethyl-N-ethylcarboxamidocholesterol) have been prepared and used for transfection (Gao et al. Biochem. Biophys. Res. Comm. 179, 280 (1991)). In another example 1,4-bis(3-N-oleylamino-propyl)piperazine was combined with histone H1 to generate a delivery reagent that was reported to be less toxic than other reagents (Wolf et al. BioTechniques 23, 139 (1997); U.S. Pat. No. 5,744,335). Several reagents are commercially available. Some examples include Lipofectin® (DOTMA:DOPE) (Invitrogen, Carlsbad, Calif.), LipofectAmine™ (DOSPA:DOPE)(Invitrogen), LipofectAmine2000™ (Invitrogen) Fugene®, Transfectam® (DOGS), Effectene®, and DC-Chol.

There are numerous disadvantages associated with the use of cationic lipids, however. For example, none of these reagents can be used universally for all cells, and many of the reagents are completely ineffective for a wide variety of cells, including primary cells. This is perhaps not surprising in light of the varying compositions of the different types of cell membranes, as well as the different barriers that can restrict entry of extracellular material into cells. Moreover, the mechanism by which cationic lipids deliver nucleic acids into cells is not clearly understood, and the reagents are less efficient than viral delivery methods and are toxic to cells, although the degree of toxicity varies from reagent to reagent.

Many biological materials are taken up by cells via receptor-mediated endocytosis, in which a ligand binds to a cell-surface receptor, leading to clustering of ligand-bound receptors, and formation of coated pits followed by internalization of the ligands into endosomes. Both enveloped viruses, like influenza virus and alphaviruses, and non-enveloped viruses, like Adenovirus, infect cells via endocytotic mechanisms. See: Pastan, I. et al. in “Virus Attachment and Entry into Cells”, (Crowell, R. L. and Lonberg-Holm, K., eds. (1986)) Am. Soc. Microbiology, Washington, p. 141-146; Kielian et al., “Entry of Alphaviruses” in The Togaviridae and Flaviviridae, (Schlesinger, S. and Schlesinger, M. J., eds. (1986)) Plenum Press, New York p.91-119; FitzGerald et al. Cell 32:607-617 (1983)). Enhancement of dendrimer-mediated transfection of some cells by chloroquine (a lysosomotropic agent) suggests that endocytosis is involved in at least some transfections.

Introduction of foreign DNA sequences into eukaryotic cells mediated by viral infection generally is several orders of magnitude more efficient than transfection with anionic lipids, cationic lipid, polyethyleneimine (PEI), peptides, or dendrimer transfection agents. In fact, viral infection of all cells in a particular culture typically requires fewer than 10 virus particles per cell. The detailed mechanism of viral fusion is not fully understood and varies between viruses, but seems to involve specific fusogenic agents, such as viral proteins, viral spike glycoproteins and peptides of viral spike glycoproteins. Cell binding and internalization also can be enhanced, accelerated or made selective with peptides that bind cell receptors. For example, the penton-base protein of the Adenovirus coat contains the peptide motif RGD (Arg-Gly-Asp) which mediates virus binding to integrins and viral internalization via receptor-mediated endocytosis (Wickham et al. Gene Therapy 2:750-756 (1995)).

The efficiency of cationic lipid transfections has been shown to be enhanced by the addition of whole virus particles to the transfection mixture. Certain viral components may also enhance the efficiency of cationic lipid-mediated transfection. For example, Kamata et al. (Nucl. Acids Res. 22:536 (1994)) suggested that “Lipofectin™”-mediated transfections may be enhanced 3-4-fold by adding influenza virus hemagglutinin peptides to the transfection mixture. Antibodies have been shown to enhance cationic lipid transfections (Trubestsky, et al, Biochim. Biophys. Acta 1131,311-313(1992)) and transferrin-poly lysine or asialoglycoprotein polylysine have been shown to enhance cationic lipid transfection (Mack et al, (1994) Am J Med Sci. 138-143.

Nevertheless, these methods do not work for all cell types, require relatively complex protocols and are inconvenient. It is apparent, therefore, that new and improved methods for introducing macromolecules, and particularly nucleic acids, into cell, are greatly to be desired. In particular, improved methods for introducing nucleic acids into a wider variety of cells, and particularly into primary cells, are greatly to be desired.

### SUMMARY OF THE INVENTION

It is therefore a goal of the invention to provide new and improved methods for introducing macromolecules into cells.

It is another goal of the invention to provide complexes, and kits for making these complexes, for use in these new and improved methods.

In accordance with these goals there has been provided a method for introducing a nucleic acid, protein, or peptide into a cell, in which a cell is contacted with a complex containing the nucleic acid, protein, or peptide, a transfection agent, and a peptide derived from a CDR of a specific antibody, where the peptide comprises a sequence comprising at least 6 contiguous amino acids from a CDR2 or CDR3 sequence of the antibody and where the peptide is functionally linked to a polycationic moiety, and where the peptide binds to the same target as the antibody. In one embodiment, the peptide may be derived from a CDR of a specific antibody, where the peptide comprises a modified amino acid sequence having at least 80% sequence identity to an unmodified sequence of at least 7 contiguous amino acids from a CDR2 or CDR3 sequence of the antibody and where the peptide is functionally linked to a polycationic moiety, and where the modified peptide retains the ability to bind the target recognized by the unmodified peptide. The polycationic moiety may contain a sequence of amino acids, where the polycationic sequence has a charge of at least +8—for example, the polycationic sequence may contain at least 8 lysine, arginine, or ornithine residues and/or the polycationic sequence is a polylysine, polyornithine, or polyarginine sequence, or a mixture of lysine, ornithine and/or arginine. The peptide may also contain a nuclear localization sequence. In one embodiment, the polycationic moiety is covalently linked to the peptide. In a particular embodiment, the polycationic moiety may be a polycationic peptide sequence, a polyamine, a peptide nucleic acid, spermine, spermidine or carboxyspermidine. In some embodiments, the peptide may comprise an amino acid sequence having at least 80% or at least 90% sequence identity to a sequence selected from the group consisting of: YYCDIRLRDP, NSRDSSGIQNV, AAWDDSLGI, VELDSFDY, DYYCAAWDDSLNGYSVF, YYCQQRSSYPYTFGG, YYCLQSMEDPYTFGG, YYCARSDGNYGYYYALDYDY, AARSPSYYRYDYGPYYAMDYD, and YYCQQSYSTPWTFGQGTK. The transfection agent may be a cationic lipid and/or a polycationic lipid and the complex may also contain a transfection enhancing agent. Suitable such agents include a nuclear localization protein or peptide.

In accordance with other embodiments, complexes as described above are provided.

In these complexes and methods the complex may contain a nucleic acid. The nucleic acid may be a vector, such as an expression vector. The expression vector may encode a structural protein, for example, an antibody or reporter protein.

The methods may be used on cells such as suspension cells.

Further provided are kits containing a transfection agent, and a peptide derived from a CDR of a specific antibody, where the peptide comprises an amino acid sequence having at least 80% sequence identity to a sequence of at least 7 contiguous amino acids from a CDR2 or CDR3 sequence of the antibody and where the peptide is functionally linked to a polycationic moiety. The transfection agent may be a cationic lipid transfection agent. The kit may also contain a peptide containing a nuclear localization sequence.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION

Compositions and methods for delivery of macromolecules into eukaryotic cells are provided. The compositions and methods are effective in a wide variety of cells, and provide a surprisingly high efficiency of transfection. More specifically, novel peptides derived from antibody complementarity determining regions (CDRs) that enhance delivery of macromolecules into cells, particularly when used in combination with cationic lipids, are provided. The peptides advantageously are combined with cationic lipids, and compositions of cationic lipids associated with enhancer elements, to provide reagents that can complex with macromolecules such as nucleic acids, proteins and peptides and permit introduction of these macromolecules into a variety of cells and tissues in vitro or in vivo with greatly enhanced efficiency compared to other lipid-based reagents. Methods for delivering macromolecules into target cells and tissues using the lipids and enhancer elements are provided.

### CDR Peptides

It has previously been shown that some synthetic peptides having sequences derived from antibody CDRs retain the ability to bind antigen even though the peptides lack the polypeptide scaffold that usually supports the CDR peptide in the intact antibody. See, for example, Laune et al., J. Biol. Chem. 272, 30937 (1997). It also has been shown that a peptide containing both the CDR2 and CDR3 regions from a polyreactive anti-DNA antibody was able to translocate a plasmid into cells when the peptide was linked to a DNA-binding lysine tail. Avrameas et al., Proc. Nat'l Acad. Sci USA 95:5601 (1998). It was noted, however, that while the peptide containing both CDR sequences was able to bind DNA and also was polyreactive to other antigens, the individual CDR peptides lacked the ability to bind DNA antigen. Moreover, although the antibody used as the source of the CDR peptides was polyreactive, i.e. was not specific to a particular antigen, the ability to deliver a plasmid into cells appeared to be related to the ability to bind DNA, an intracellular antigen, and was therefore not dependent on the ability to bind an extracellular antigen.

Other workers have described experiments where peptides containing CDR2 sequences of an anti-reovirus antibody retained the ability to be bound by reovirus (Williams et al., Proc. Nat'l Acad. Sci USA 85:6488 (1988)) and a peptide derived from the VH CDR3 of an antibody that bound the platelet fibrinogen receptor contained a fibrinogen-like sequence that bound the fibrinogen receptor (Taub et al., J. Biol. Chem. 264:259 (1988)). A systematic study of 12-residue peptides derived from CDR regions and surrounding regions of three different antibodies found several peptides that retained antigen-binding activity, although it was observed that in several cases the most active peptides did not contain the most CDR residues. Laune et al., J. Biol. Chem. 272:30937 (1997). Finally, it has been proposed that “molecular recognition units” can be obtained by taking CDR peptides from IgM antibodies, where the peptide sequence is obtained from an IgM antibody where the antibody gene sequence is one that has been rearranged in only one CDR when compared to that of germline genes. See U.S. Pat. No. 5,196,510.

Other methods of identifying peptides that specifically bind a specific target are known. The best known of these is the phage display method developed by Smith, where peptides are displayed on the surface of filamentous phage and can be selected by screening against a binding partner. The sequence of the binding peptide can then be determined by sequencing the DNA insert that encoded the displayed peptide. Smith, Science 228:1315 (1985).

The present inventors have surprisingly found that peptides derived from the CDR regions, and advantageously the CDR2 and, especially, the CDR3 regions of antigen-specific antibodies, can be prepared and used in combination with cationic lipids to deliver nucleic acid into cells at a much higher efficiency than prior methods. Moreover, and even more surprisingly, the CDR peptides are significantly more effective for transfection of exogenous nucleic acids into cells than cell-binding peptides identified by phage display methods, even though the phage display-derived peptides apparently bind tightly and specifically to cellular targets. The CDR-derived peptides are particularly, and surprisingly, effective at facilitating transfection of cells that are difficult to transfect using conventional reagents, such as primary fibroblasts and HepG2 cells.

Design of the CDR Peptides

To design a CDR peptide that will be useful for facilitating delivery of exogenous DNA into a desired eukaryotic cell type, antibodies are identified that bind to antigenic proteins that are expressed by the target cell. Advantageously, the antigenic proteins, carbohydrates, or lipids are expressed on the surface of the cell. More advantageously, antibodies that are specific for the protein antigen are identified. If a specific antibody for the desired antigen is not available, antibodies can be generated using methods that are well known in the art. For example, murine monoclonal antibodies can be raised. See “Antibodies: A Laboratory Manual” Harlow and Lane, CHSL Press 1999. Alternatively, antibodies can be obtained from display libraries, such as libraries of scFv or Fab antibodies displayed on the surface of filamentous phage.

Once a suitable specific antibody has been identified, the CDR sequences are identified. In some instances, such as an antibody that has been characterized in the scientific literature, the CDR sequences already have been identified. In other circumstances, it is necessary to obtain the protein sequence of the antibody and identify the CDR regions using methods that are known in the art. For example, for an antibody produced by a hybridoma, methods for identifying the antibody coding sequence from the hybridoma are well known in the art. For antibodies from a phage display library, methods for obtaining the antibody sequence by sequencing the nucleic acid insert or phagemid from the phage also are well known in the art. Once the primary amino sequences of the antibody heavy and light chains have been identified, methods for identifying the CDR regions are known in the art.

Peptides that contain some or all the amino acid sequence of the antibody CDRs can then be prepared. Advantageously, the CDR2 and/or CDR3 sequences are used to prepare the peptides. More advantageously, the peptides contain at least 6 contiguous amino acids from the CDR2 or CDR3 sequences. It is not typically necessary to include all the amino acids from the CDR regions provided that at least 6 contiguous amino acids from the CDR are present. When the peptide is derived from the CDR3 of the VH domain of the antibody, advantageously a tripeptide sequence YYC may be added to the C-terminus of the peptide. The YYC motif typically is present in the framework region C-terminal to the VH CDR3 and is used to identify the location of the CDR3. The YYC tripeptide may be added to the C-terminus of the peptide even without including all the amino acid residues that follow the YYC sequence in the VH CDR3.

Once the appropriate CDR sequence has been identified, a synthetic peptide is prepared that also has the ability to complex with negatively charged macromolecules such as nucleic acids. To achieve this, the peptide may be functionally linked to a polycationic moiety. The functional linkage may be a covalent linkage or may be non-covalent. An example of a non-covalent linkage between the peptide and the polycationic moiety binding moiety is where the peptide contains a first member of a binding pair, and the polycationic moiety contains a second member of the binding pair, where association of the first and second members of the binding pair results in functional linkage of the CDR peptide and the polycationic moiety. Suitable binding pairs include an antibody and an antigen, streptavidin/biotin, and the like. The polycationic moiety may be, for example, a polycationic peptide sequence, a polyamine, a peptide nucleic acid, spermine, spermidine or a carboxyspermidine, although the skilled the artisan will recognize that other polycationic moieties can be used.

In one embodiment, the polycationic moiety is a polycationic peptide sequence that is covalently linked in a single peptide chain with the CDR peptide sequence. The polycationic peptide may contain a plurality of lysine, ornithine, arginine, or other positively charged amino acid residues, although typically a polylysine moiety is used. The polycationic moiety may be linked to the N-terminus, the C-terminus, or to the side chain of an amino acid that is internal to the CDR peptide sequence. In alternative embodiments, the CDR peptide can be covalently linked to a polycationic lipid directly, or indirectly. For example, indirect linkage can be achieved where the peptide is linked to one member of a binding pair, such as biotin, and a lipid can be linked to the corresponding member of the binding pair, such as avidin/streptavidin.

The CDR peptides of the present invention may be bounded by further amino acid sequences on either or both of the N- and C-termini of the CDR amino acid sequence. Accordingly the CDR sequence may be N-terminal, C-terminal, or internal within the peptides. The CDR sequences within the peptides are generally less than 30 amino acids in length. As described above, the CDR peptide sequence may be bounded on either side by sequences derived from antibody frameworks and these framework residues are included within the 30 amino acid length limitation. Typically no more than 10 framework residues are included within the CDR peptide sequence. The skilled artisan will recognize that peptides that contain close sequence variants to the CDR sequence also can be effective for promoting delivery of macromolecules into cells. Accordingly, the present invention also contemplates CDR peptides that contain sequences that have at least 80% or at least 90% sequence identity with the CDR sequences and, specifically, with the sequences disclosed below.

The present inventors have identified a series of CDR peptides that are highly and surprisingly effective at promoting transfection when used in transfection complexes with cationic lipids:

YYC DIR LRD P
NSR DSS GIQ NV
AAW DDS LGI
VEL DSF DY
DYY CAA WDD SLN GYS VF
YYC QQR SSY PYT FGG
YYC LQS MED PYT FGG
YYC ARS DGN YGY YYA LDY DY
AAR SPS YYR YDY GPY YAM DYD
YYC QQS YST PWT FGQ GTK.

Each of the peptides was prepared with a polycationic peptide fused C-terminal or N-terminal to the sequence shown. Typically, a 8-20 residue polylysine or polyarginine sequence is used, although the skilled artisan will recognize that a wide variety of polycationic sequences can be used.

The CDR peptide may also contain a nuclear localization sequence (NLS) that promotes delivery of the peptide and molecules bound to it to the nucleus of a cell. Suitable nuclear localization sequences are well known in the art and include, for example, the well-known SV40 sequence. In another embodiment, a separate NLS peptide may be used to promote intracellular delivery in conjunction with the CDR peptide. Suitable NLS peptides are known in the art and include PKKKRKVEDPYC, PKKKRKV, KKKRKVC, GKKRSKA, KRPRP, GNKAKRQRST, GGAAKRVKLD, SALIKKKKKMAP, RKLKKLGN, PQPKKKP, ASKSRKRKL, KKKYK, KKKYKC, KSKKK, KRVKLC, and AKRVKL.

### Transfection Complexes

Advantageously, the CDR peptide is used as part of a transfection complex. In one embodiment, a transfection complex provided herein contains the macromolecule that is to be delivered to the cell, the CDR peptide as described above, and a transfection agent. The complex is formed by mixing the reagents in any order and is then added to the cells to be transfected. In certain illustrative aspects, transfection complexes are formed using complexes that include a CDR peptide, and a transfection reagent.

In still other embodiments, the complex may also contain a transfection enhancing agent that facilitates entry of the complex into the target cell or that facilitates subcellular or cellular targeting of the complex. Exemplary transfection enhancing agents include nuclear localization peptides, another CDR peptide or protein, a ligand for a cell-surface receptor, endosomal release agent, or membrane penetration proteins or peptides and the like, as described in more detail below. In one example, the transfection enhancing agent is the PLUS™ Reagent (available from Invitrogen Corporation, Carlsbad, Calif.)).

In other embodiments, other peptides, proteins, fragment thereof, or modified peptides, proteins and fragments thereof that promote still more efficient transfection may be used along with or as components of the complexes of the invention. In one embodiment these peptides, proteins or fragments thereof are bound or added to the nucleic acid prior to adding the complex, while in other embodiments the peptides, proteins, or fragments thereof may be added or complexed with the complex prior to addition of the nucleic acid. Alternatively, the nucleic acid may be combined with the complex prior to addition of the peptide, protein, etc.

### Transfection Agents

An additional component of the complexes used in the present invention is a transfection agent. Suitable transfection agents in the context of the present invention include cationic and polycationic polymers or particles, and/or cationic and polycationic lipids. Cationic and polycationic polymers suitable for use in the invention are known in the art and include, for example, dense star dendrimers, PAMAM dendrimers, NH3 core dendrimers, ethylenediamine core dendrimers, dendrimers of generation 5 or higher, dendrimers with substituted groups, dendrimers comprising one or more amino acids, grafted dendrimers and activated dendrimers, polyethyleneimine, polyethyleneimine conjugates, and polyalkylenimine. The skilled artisan will recognize that the present invention is not limited to use of these polycationic polymer transfection agents.

Advantageously, the transfection agent is a lipid, preferably a cationic lipid (or a mixture of a cationic lipid and neutral lipid). This lipid can be used to form a peptide- or protein-nucleic acid-lipid aggregate which facilitates introduction of the anionic nucleic acid through cell membranes, including the nuclear membrane. Transfection compositions of this invention comprising peptide- or protein-nucleic acid complexes and lipid can further include other non-peptide agents that are known to further enhance transfection.

Inclusion of a peptide- or protein-nucleic acid complex or a modified peptide- or protein-nucleic acid complex in a cationic lipid transfection composition can significantly enhance transfection (often by 2-fold or more, and in some cases by over 30 fold) of the nucleic acid compared to transfection of the nucleic acid mediated by the cationic lipid alone. Enhancement of polycationic polymer transfection by CDR peptides is pronounced in a wide variety of cell lines, including human primary cell lines and in cell lines that are generally considered by those in the art to be “hard-to-transfect.”

Monovalent or polyvalent cationic lipids are employed in cationic lipid transfecting compositions. Illustrative monovalent cationic lipids include DOTMA (N-[1-(2.3-dioleoyloxy)-propyl]-N,N,N-timethyl ammonium chloride), DOTAP (1,2-bis(oleoyloxy)-3-3-(trimethylammonium)propane), DMRIE (1,2-dimyristyloxypropyl-3-dimethyl-hydroxy ethyl ammonium bromide), DDAB (dimethyl dioctadecyl ammonium bromide), DC-Chol (3-(dimethylaminoethane)-carbamoyl-cholestrerol). Preferred polyvalent cationic lipids are lipospermines, specifically, DOGS (Dioloctadecylaminoglycyl spermine), DOSPA (2,3-dioleyloxy-N-[2(sperminecarboxamido)ethyl]-N,N-dimethyl-1-propanamin-ium trifluoroacetate) and DOSPER (1,3-dioleoyloxy-2-(6carboxy spermyl)-propyl-amid; N-1-dimethyl-N-1-(2,3-dialkyloxypropyl)-2-hydroxypropane-1,3-diamine including but not limited to N-1-dimethyl-N-1-(2,3-diaoleoyloxypropyl)-2-hydroxypropane-1,3-diamine, N-1-dimethyl-N-1-(2,3-diamyristyloxypropyl)-2-hydroxypropane-1,3-diamine, N-1-dimethyl-N-1-(2,3-diapalmityloxypropyl)-2-hydroxypropane-1,3-diamine; N-1-dimethyl-N-1-(2,3-dialkyloxypropyl)-2-(3-amino-2-hydroxypropyloxy)propane-1,3-diamine including but not limited to N-1-dimethyl-N-1-(2,3-diaoleoyloxypropyl)-2-(3-amino-2-hydroxypropyloxy)propane-1,3-diamine, N-1-dimethyl-N-1-(2,3-diamyristyloxypropyl)-2-(3-amino-2-hydroxypropyloxy)propane-1,3-diamine, N-1-dimethyl-N-1-(2,3-diapalmityloxypropyl)-2-(3-amino-2-hydroxypropyloxy)propane-1,3-diamine; and the di-and tetra-alkyl-tetra-methyl spermines, including but not limited to TMTPS (tetramethyltetra-palmitoyl spermine), TMTOS (tetramethyltetraoleyl sp. ermine), TMTLS (tetramethlytetralauryl spermine), TMTMS (tetramethyltetramyristyl spermine) and TMDOS (tetramethyldioleyl spermine); and 1,4,-bis[(3-amino-2-hydroxypropyl)-alkylamino]-butane-2,3-diol including but not limited to 1,4,-bis[(3-amino-2-hydroxypropyl)-oleylamino]-butane-2,3-diol, 1,4,-bis[(3-amino-2-hydroxypropyl)-palmitylamino]-butane-2,3-diol, 1,4,-bis[(3-amino-2-hydroxypropyl)-myristylamino]-butane-2,3-diol; and 1,4-bis(3-alkylaminopropyl)piperazine including but not limited to 1,4-bis[(3-oleylamino)propyl]piperazine, 1,4-bis[(3-myristylamino)propyl]piperazine, 1,4-bis[(3-palmitylamino)propyl]piperazine; and a 1,4-bis[(3-(3-aminopropyl)-alkylamino)propyl)piperazine including but not limited to 1,4-bis[(3-(3-aminopropyl)-oleylamino)propyl]piperazine, 1,4-bis[(3-(3-aminopropyl)-myristylamino)propyl]piperazine, 1,4-bis[(3-(3-aminopropyl)-palmitylamino)propyl]piperazine; and 1,4-bis[(3-(3-amino-2-hydroxypropyl)-alkylamino)propyl]piperazine including but not limited to 1,4-bis[(3-(3-amino-2-hydroxypropyl)-oleylamino)propyl]piperazine, 1,4-bis[(3-(3-amino-2-hydoxypropyl)-myristylamino)propyl]piperazine, 1,4-bis[(3-(3-amino-2-hydroxypropyl)-palmitylamino)propyl]piperazine, 1,4-bis[(3-(3-aminopropyl)-alkylamino)-2-hydroxypropyl]piperazine including but not limited to 1,4-bis[(3-(3-aminopropyl)-oleylamino)-2-hydroxy-propyl]piperazine, I,4-bis[(3-(3-aminopropyl)-myristylamino)-2-hydroxypropyl]piperazine, 1,4-bis[(3-(3-aminopropyl)-palmitylamino)-2-hydroxy-propyl]piperazine

In certain illustrative examples the cationic lipids that may be used include the commercial agents LipofectAmine™ 2000, LipofectAmine™, Lipofectin®, DMRIE-C, CellFectin®(Invitrogen), Oligofectamine®(Invitrogen), LipofectAce® (Invitrogen), Fugene® (Roche, Basel, Switzerland), Fugene® HD (Roche), Tranffectam® (Tranfectam, Promega, Madison, Wis.), Tfx-10® (Promega), TN-20® (Promega), Tfx-50® (Promega), Transfectin™ (BioRad, Hercules, Calif.), SilentFect™ (Bio-Rad), Effectene® (Qiagen, Valencia, Calif.), DC-chol (Avanti Polar Lipids), GenePorter® (Gene Therapy Systems, San Diego, Calif.), DharmaFect I® (Dharmacon, Lafayette, Colo.), DharmaFect 2® (Dharmacon), DharmaFect 3® (Dharmacon), DharmaFect 4® (Dharmacon), Escort™ III (Sigma, St. Louis, Mo.) and Escort™ IV (Sigma).

Cationic lipids are optionally combined with non-cationic lipids, particularly neutral lipids, for example lipids such as DOPE (dioleoylphosphatidylethanolamine), DPhPE (diphytanoylphosphatidylethanolamine) or cholesterol. The ratio can vary from 1:1 (molar) to 4:1 (molar) of cationic to neutral lipids. Transfection properties of cationic lipid compositions composed of a 0.5:1 to 4:1 mixtures of 1,4-bis[(3-(3-aminopropyl)-oleylamino)propyl]piperazine and DOPE and a 1:1 to 4:1 mixtures of 1,4-bis[(3-(3-aminopropyl)-oleylamino)propyl]piperazine and cholesterol as well as a 1:1 to 4:1 mixtures of 1,4-bis[(3-(3-aminopropyl)-palmitylamino)propyl]piperazine and DOPE and a 1:1 to 4:1 mixture of 1,4-bis[(3-(3-aminopropyl)-palmitylamino)propyl]piperazine and cholesterol are significantly enhanced by peptides and proteins of the invention.

Transfection properties of cationic lipid compositions composed of a 1:1 to 4:1 mixtures of 1,4-bis[(3-(3-amino-2-hydroxypropyl)-oleylamino)propyl]piperazine and DOPE and a 1:1 to 4:1 mixtures of 1,4-bis[(3-(3-amino-2-hydroxypropyl)-oleylamino)propyl]piperazine and cholesterol as well as a 1:1 to 4:1 mixtures of 1,4-bis[(3-(3-amino-2-hydroxypropyl)-palmitylamino)propyl]piperazine and DOPE and a 1:1 to 4:1 mixture of 1,4-bis[(3-(3-amino-2-hydroxypropyl)-palmitylamino)propyl]piperazine and cholesterol are significantly enhanced by peptides and proteins of the invention.

A cationic lipid composition composed of a 3:1 (w/w) mixture of DOSPA and DOPE or a 1:1 (w/w) mixture of DOTMA and DOPE is generally useful in transfecting compositions of this invention, although it will be appreciated that many other compositions can be used. Preferred transfection compositions are those which induce substantial transfection of a higher eukaryotic cell line. Inclusion of a peptide- or protein-nucleic acid or modified peptide- or protein-nucleic acid complex in a polycationic polymer transfection composition can significantly enhance transfection (by 2-fold or more) of the nucleic acid compared to transfection of the nucleic acid mediated by the polycationic polymer (e.g. dendrimer) alone or in combination with DEAE-dextran or chloroquine or both.

Enhancement of transfection by peptides, proteins, modified peptides or modified proteins is pronounced in a wide variety of cell lines, in cell lines that are generally considered by those in the art to be “hard-to-transfect.”

Transfection Enhancing Agents

The complexes formed between the CDR peptide, the optional polycationic moiety, the macromolecule and the transfection agent may be further enhanced by inclusion of moieties such as proteins or peptides that function for nuclear or other sub-cellular localization, function for transport or trafficking, are receptor ligands, comprise cell-adhesive signals, cell-targeting signals, cell-internalization signals, endocytosis signals, or cell penetration signals as nucleic acid sequences encoding one or more protein chains. Where the protein produced is a pharmaceutical product, the protein can be formulated accordingly, for example in an appropriate choice of physiologic medium.

The transfection composition provided herein can also be used to introduce peptides and proteins and the like into cells using methods that are known in the art. Methods of using cationic lipids for peptide and protein delivery previously have been described. In addition, the transfection compositions can be used to deliver nucleic acids, peptides and proteins and the like into tissues in vivo. Methods of using lipids for delivering compounds to tissue in vivo previously have been described. The transfection compositions can, with appropriate choice of physiologic medium, be employed in therapeutic and diagnostic applications.

The macromolecules which can be delivered into cells include, but are not limited to, nucleic acids. The nucleic acid can be any type of nucleic acid that presently is known or that may be prepared or identified in the future, provided that the nucleic acid is sufficiently negatively charged to form a lipid aggregate, liposome, or liposome-like complex when admixed with the lipid. Nucleic acid, as used herein, refers to deoxyribonucleotides or ribonucleotides and mixtures and polymers thereof in single- or double-stranded form. The term encompasses nucleic acids containing known nucleotide analogs or modified backbone residues or linkages, which are synthetic, naturally occurring, and non-naturally occurring, which have similar binding properties as a reference nucleic acid, and which are metabolized in a manner similar to a reference nucleotides. Examples of such analogs include, without limitation, phosphorothioates, phosphoramidates, methyl phosphonates, chiral-methyl phosphonates, 2-0-methyl ribonucleotides, peptide-nucleic acids (PNAs). The nucleic acid may be in the form of an antisense molecule, for example a “gap-mer” containing an RNA-DNA-RNA structure that activates RNAseH. The nucleic acid can be, for example, DNA or RNA, or RNA-DNA hybrid, and can be an oligonucleotide, plasmid, parts of a plasmid DNA, pre-condensed DNA, product of a polymerase chain reaction (PCR), vectors, expression cassettes, chimeric sequences, chromosomal DNA, or derivatives of these groups or other form of nucleic acid molecule. The nucleic acid may be a double-stranded RNA molecule of the type used for inhibiting gene expression by RNA interference. The nucleic acid may be a short interfering double stranded RNA molecule (siRNA). The nucleic acid molecule can also be a Stealth™ RNAi molecule (Invitrogen Corporation, Carlsbad, Calif.).

Accordingly, provided herein is a method of introducing macromolecules into cells. An exemplary method includes forming a lipid-nucleic acid complex using a lipid, a nucleic acid and an enhancing agent, as described herein, and contacting cells, such as, by way of example only, eukaryotic cells, with such a complex. The lipid may be in the form of a lipid-aggregates, including, but not limited to, liposomes. It will be understood that incubation times, mixing protocols, and other specific aspects of the methods of the invention can be optimized using methods known in the art.

Cells which can be transfected according to the such methods include, but are not limited to, virtually any eukaryotic cell including primary cells, cells in culture, a passaged cell culture or a cell line, and cells in cultured tissue. Suitable cells include human cell lines and animal cell lines. The cell may be a fibroblast. The cells can be attached cells or cells in suspensions. In certain illustrative aspects, the cells are suspension CHO-S cells and suspension 293-F cells. Other cells that may be used include, without limitation, 293, 293-S, CHO, Cos, 3T3, Hela, primary fibroblasts, A549, Be2C, SW480, CHOK1, Griptite 293, HepG2, Jurkat, LNCap, MCF-7, NIH-3T3, PC12, C6, Caco-2, COS-7, HL60, HT-1080, IMR-90, K-562, SK-BR3, PHP1,HUVEC, MJ90, NHFF, NDFF and primary neurons.

In another embodiment is a method for producing a protein which includes contacting a cell with a lipid-nucleic acid complex as described above, where the nucleic acid encodes the protein. The cells are incubated to produce the protein and the protein is collected. Cells which can be used for protein production are described above. In addition, any composition which includes a lipid and a CDR element can be used for transfection of cells. Such compositions are further discussed herein, and include, but are not limited to compositions comprising lipids, a co-lipid and an optional transfection enhancing agent such as a CDR peptide or protein.

The lipid aggregates of the present invention form a complex when they come in contact with macromolecules such as nucleic acids. The lipids optionally may be used in slight excess and, in such as case, may form a cationic complex. Without being bound by any theory, it is thought that cationic complexes are attracted to the cell membrane thereby facilitating uptake by the cell. Such lipid aggregates include liposomes, unilamellar vesicles, multilamellar vesicles, micelles and the like, which can have particle sizes in the nanometer to micrometer range. The structure of various types of lipid aggregates varies, depending on composition and method of forming the aggregate. Methods of making lipid aggregates are known in the an, and include, but are not limited to, reverse evaporation, sonication and microfluidization.

In another embodiment, provided herein is a method for producing a protein comprising, transfecting a cell with a nucleic acid encoding the protein, incubating the cell to produce the protein, and collecting the protein, where the transfecting is performed by contacting the cell with a composition comprising a lipid, optionally with a CDR peptide. The composition for transfecting the cell can be any of the compositions provided herein, including those that include other lipids and/or additional peptides and proteins.

The lipids may also be used to introduce peptides and proteins and the like into cells using methods that are known in the art. Methods of using cationic lipids for peptide and protein delivery previously have been described.

In addition, the lipids may be used to deliver nucleic acids, peptides and proteins and the like into tissues in vivo. Methods of using lipids for delivering compounds to tissue in vivo previously have been described.

Cationic lipid compositions composed of 1,4-bis[(3-(3-aminopropyl)-alkylamino)propyl)piperazine lipids and neutral lipids, including 1:1 to 4:1 mixtures of 1,4-bis[(3-(3-aminopropyl)-oleylamino)propyl]piperazine and DOPE and a 1:1 to 4:1 mixtures of 1,4-bis[(3-(3-aminopropyl)-oleylamino)propyl]piperazine and cholesterol as well as a 1:1 to 4:1 mixtures of 1,4-bis[(3-(3-aminopropyl)-palmitylamino)propyl]piperazine and DOPE and a 1:1 to 4:1 mixture of 1,4-bis[(3-(3-aminopropyl)-palmitylamino)propyl]piperazine and cholesterol were effective at transfecting various cell types with nucleic acids

Compositions and/or Methods that Use Other Lipids

In certain illustrative examples, the lipids also can be used in compositions with other lipids and/or with additional transfection-enhancing agents to deliver macromolecules. Such compositions may contain a lipid and a co-lipid which is neutral, positively charged (such as a cationic lipid) or negatively charged. Such neutral lipids include, but are not limited to, diacylphosphatidylcholine, diacylphosphatidylethanolamine (DOPE), ceramide, sphingomyelin, cephalin, cholesterol, cerebrosides and diacylglycerols. for example. The cationic lipids include, but are not limited N,N-dioleyl-N,N-dimethylammonium chloride (DODAC); N-(2,3-dioleyloxy)propyl)-N,N, N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3 -(N-(N′,N′-dimethylaminoethane)-carbamoyl)cholesterol (DC-Chol); N-(1,2-dimyristyloxyprop-3-yl)-N,N-dimethyl-N-hydroxyethyl ammonium bromide (DMRIE); 1,2-Dilinoleyloxy-N,N-dimethylaminopropane (DLinDMA); 1,2-Dilinolenyloxy-N,N-dimethylaminopropane (DLenDMA), DODAP, DODMA, and DMDMA. The cationic lipids may also include, but are not limited to, LipofectAmine™ 2000, LipofectAmine™, Lipofectin®, DMRIE-C, Fugene®, Fugene® HD, Transfectam®, Transfectin™, SilentFect™, and Effectene®. The anionic lipids include, but are not limited to, phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

Transfection enhancing agents which may be included in the compositions described above include, but are not limited to, transfection-enhancing peptides or proteins that function to deliver the macromolecule to specific sub-cellular locations such as the nucleus or other organelles, that function for cellular transport or trafficking, that are receptor ligands, that comprise cell-adhesion signals, cell-targeting signals, cell-internalization signals or endocytosis signals. Other examples include peptides or functional portions thereof that are enveloped or non-enveloped viral proteins or derived from enveloped or non-enveloped viral proteins, that are enveloped or non-enveloped viral fusogenic proteins or derived from enveloped or non-enveloped viral fusogenic proteins, that contain viral nuclear localization signals, that are receptor-ligands, that contain cell adhesion signals, cell-targeting signals, and/or internalization- or endocytosis-triggering signals.

In an illustrative example, the lipids of the present invention can be used in conjunction with Plus Reagent™, as provided in exemplary transfection protocols provided herein. Furthermore, the lipids can be formulated in liposomal or non-liposomal formulations, that can include a helper lipid, such as DOPE, along with a CDR peptide, as described above, and used in conjunction with Plus Reagent™ to deliver nucleic acids to cells. Exemplary peptides or proteins that may be used in combination with the lipids include NLS containing proteins such as histones, transferring, reovirus fusion peptides and fragments thereof.

The lipids may be formulated with one or more nucleic acids into liposomes or liposome-like vehicles in the presence or absence of co-lipid such as, by way of example only, dioleylphosphatidyl ethanolamine (DOPE) or cholesterol. The lipids may be formulated into liposomes, for example using the method of reverse evaporation, which is well known in the art. Alternatively the lipids may be formulated by other well known methods for liposome formation such as sonication, microfluidization etc. These liposome formulations are effective for transfecting DNA into cultured cells.

In one method, a nucleic acid is contacted with a CDR peptide and the resulting mixture is added to a mixture of a cationic lipid and a neutral lipid, where the CDR peptide contains a sequence as described above, for example, and in the Examples below. In another method, a CDR peptide is contacted with a lipid followed by addition of a nucleic acid or protein capable of aggregating the peptide-or protein-nucleic acid complex.

In certain embodiments of the present invention methods involve contacting any cell, preferably a eukaryotic cell, with a transfection complex comprising at least a CDR peptide, a lipid and a nucleic acid as described above. The complex optionally may also contain one or more additional peptides or proteins, such as a membrane-permeabilizing, transport or trafficking sub-cellular-localization, or receptor-ligand peptide or protein. These additional peptides or proteins optionally may be conjugated to a nucleic acid-binding group, or optionally conjugated to a lipid where the peptide or protein or modified peptide or protein is non-covalently associated with the nucleic acid.

Without being bound by any theory, applicants believe that the complexes of the present invention are lipid aggregates that typically contain liposomal structures, although the precise nature of these structures is not presently known. Accordingly, in certain illustrative examples, complexes of the present invention are liposomal complexes. The entire complex, or a portion of the complex, such as a lipid portion, can be formulated into liposomes, for example using the method of reverse evaporation, which is well known in the art. Alternatively the lipid portion of the complex or the entire complex, can be formulated by other well known methods for liposome formation such as sonication or microfluidization. These liposome formulations are effective for transfecting DNA into cultured cells.

In one embodiment, a complex containing the CDR peptide or protein and the nucleic acid (where the CDR peptide or protein can be conjugated to a nucleic-acid binding group) is first formed and then combined with a cationic lipid for transfection. In a related embodiment, a peptide- or protein-lipid conjugate is combined optionally with other lipids, including any appropriate cationic lipid, and then combined with nucleic acid for transfection. In another related embodiment, a nucleic acid-lipid complex is formed and then combined with a CDR peptide for transfection. As discussed above, the lipid containing complexes of any of these embodiments can be liposomal or non-liposomal formulations.

The complexes and methods of the present invention, especially those involving transfection compositions that include complexes provided herein, can be used for in vitro and in vivo transfection of cells, particularly of eukaryotic cells, and more particularly to transfection of higher eukaryotic cells, including animal cells. The methods of this invention can be used to generate transfected cells which express useful gene products. For example, the methods can be used to produce recombinant antibody molecules, typically by expressing a recombinant light chain molecule and a recombinant heavy chain molecule from one or more expression vectors that are introduced into a cell, especially a suspension cell, using the complexes provided herein. The methods of this invention can also be employed as a step in the production of transgenic animals. The methods of this invention are useful as a step in any therapeutic method requiring introduction of nucleic acids into cells including methods of gene therapy and viral inhibition and for introduction of antisense or antigene nucleic acids, ribozymes, RNA regulatory sequences, siRNA, RNAi, Stealth™ RNAi (Invitrogen Corporation, Carlsbad Calif.) or related inhibitory or regulatory nucleic acids into cells. In particular, these methods are useful in cancer treatment, in in vivo and ex vivo gene therapy, and in diagnostic methods.

The transfection compositions and methods of this invention comprising peptides, proteins, peptide or protein fragments or modified peptides or modified proteins, can also be employed as research agents in any transfection of eukaryotic cells done for research purposes.

Accordingly, provided herein is a method of introducing a macromolecule into a cell, by forming a transfection composition that includes a nucleic acid and a complex comprising a lipid and a CDR peptide as described above; and contacting a eukaryotic cell with the transfection composition. The detailed Example set forth below is merely an illustrative protocol for using compositions of the present invention to transfect eukaryotic cells, but is not limiting of the present invention.

It will be understood that quantities, concentrations and volumes of complexes, complex components, and nucleic acid or other macromolecules, incubation times, mixing protocols, and other specific aspects of the methods of the invention are known in the art or can be optimized and/or identified using methods known in the art. As illustrated in the Examples section herein, volumes and concentrations of nucleic acid or other macromolecule, volume and concentration of the transfection complexes provided herein, volumes and compositions of diluents, and volume and concentration of cells, can be determined using standard experimental approaches for such optimization and titration, including, for example, methods that utilize cytotoxicity assays and/or methods that employ transfection using nucleic acid expression vectors that express reporter genes, such as beta galactosidase, luciferase, and/or fluorescent proteins. Furthermore, cell densities can be optimized using standard methods, and cell densities for transfections using the transfection complexes provided herein can range, for example, from high density >75% to low density <50%. Exemplary diluents for complex formation, for example, include reduced serum, or serum-free media, such as D-MEM and RPMI 1640 and OptiPro™, Opti-MEM® (Invitrogen Corporation). Incubation times for forming complexes can be determined using routine methods, although typical incubation times are between 5 and 240 minutes. In addition, it will be understood that media for cell culturing can be chosen based on the cell line to be transfected and based on the particular application of the method. For example, for the production of proteins in suspension cells, in illustrative embodiments, reduced serum, and preferably serum-free medium can be used. In certain illustrative embodiments, animal original free medium is employed, such as, but not limited to, 293 Expression Medium (Invitrogen Corporation) and CD-CHO Medium (Invitrogen Corporation). in certain aspects depending on the cell type to be transfected, antibiotics can be excluded from post transfection media. Incubation times for post-transfection culturing of cells vary depending upon the cells and the conditions used, but typically range from 2 hours to 7 days. For large-scale protein production, cells can be incubated, as a non-limiting example, for between 1 day and 7 days.

It will be understood that a wide range of concentrations of lipids, co-lipids and transfection enhancing agents can be used in the complexes, compositions and methods provided herein. For example, in an illustrative non-limiting example of a composition provided herein that includes a complex of a lipid and a CDR peptide, the total exemplary, non-limiting combined concentration of lipid and CDR peptide in the composition can be between 1 mg/ml and 4 mg/ml. The range of peptide added to the lipid at 1mg/ml can be between 100 pgml and 3 mg/ml. the ratio of the helper lipid to cationic lipid can be between 0.25:1.0 (molar) and pure compound.

Cells that can be transfected according to the present invention include, for example, virtually any eukaryotic cell including primary cells, cells in culture, and cells in cultured tissue. The cells can be attached cells or cells in suspensions. In certain illustrative aspects, the cells are suspension CHO-S cells and suspension 293-F cells. Other cells that can be transfected using the agents and methods of the invention include, but are not limited to, 293, such as GripTite 293 MSR (Invitrogen Corporation), CHO, Cos7, NIH3T3, Hela, primary fibroblast, A549, Be2C, SW480, Caco2, primary neurons. Jurkat, C6, THP1, IMR90, HeLa, ChoK1, GT293, MCF7, HT1080, LnCap, HepG2, PC12, SKBR3, and K562 cells.

In another embodiment, provided herein is a method for producing a protein comprising, transfecting a cell with a nucleic acid molecule encoding the protein, incubating the cell to produce the protein, and collecting the protein, where the transfecting is performed by contacting the cell with a transfection composition of the present invention. By way of example, such compositions can include the nucleic acid molecule encoding the protein, a fusion agent, and a lipid, where the fusion agent include a fusion promoting amino acid sequence.

### Pharmaceutical Compositions

Transfection agents and transfection-enhancing agents of this invention can be provided in a variety of pharmaceutical compositions and dosage forms for therapeutic applications. For example, injectable formulations, intranasal formulations and formulations for intravenous and/or intralesional administration containing these complexes can be used therapy.

In general the pharmaceutical compositions of this invention should contain sufficient transfection agent and any enhancing agents (peptide, protein, etc.) to provide for introduction of a sufficiently high enough level of nucleic acid into the target cell or target tissue such that the nucleic acid has the desired therapeutic effect therein. The level of nucleic acid in the target cell or tissue that will be therapeutically effective will depend on the efficiency of inhibition or other biological function and on the number of sites the nucleic acid must affect.

The present invention, thus generally described, will be understood more readily by reference to the following examples, which are provided by way of illustration and are not intended to be limiting of the present invention.

### EXAMPLES

### Methods

Transfection of, NIH3T3, HepG2, Human Fibroblast, Jurkat and Cos-7 and with β-galactosidase reporter plasmid pCMVSPORT-β-gal was carried out as follows:

Cells were plated in 96-well plates with 100 μl of media containing 10% fetal calf serum the day before transfection such that a desired confluency (70%-95%) was achieved the following day. The following day DMS lipid (1:1 DOPE) and DNA/peptide were mixed in Opti-MEM to form DNA/lipid/peptide complexes. Complexes were formed by adding various amounts of lipid (1-6 μl) to 100 μl of Opti-MEM. Plasmid DNA (1.0 μg) was added to 100 μl Opti-MEM then 1.0 ug of each peptide was added to the DNA mixture and incubated for 10 minutes. The DNA/peptide and lipids solutions were then mixed to form DNA/peptide/lipid complexes. The complexes were incubated for an additional 15 minutes. After incubation 20 μl of each of the resulting complexes was added directly to the cells in 10% serum. Cells were incubated for an additional 16-24 hours to allow expression of the plasmid. Medium was removed and the cells were lysed in 100-200 μl of lysis buffer. The lysates (20 μl) were assayed for β-gal activity using the enzymatic substrate ONPG. Total activity was determined by reading the OD at 405 nm using a Bio-Rad Benchmark Microplate Spectrophotometer.

The following results were obtained using this protocol:

Fold Ehancement over Lipid Alone

| | | | | Human | |
|---|---|---|---|---|---|
| | | | NIH | Fibro- | |
| | PC12 | Jurkat | 3T3 | blast | HepG2 |
| Lipid No | 0 | 0 | 0 | 0 | 0 |
| Peptide | | | | | |
| Lipid with | 1.8 | 4.56 | 1.7 | 3.7 | 0.97 |
| Peptide #1 | | | | | |
| Lipid with | 2.09 | 4.8 | 0.89 | 14.5 | 1.3 |
| Peptide #2 | | | | | |
| Lipid with | 2.88 | 4.5 | 1.5 | 12.9 | 1.9 |
| Peptide #3 | | | | | |
| Lipid with | 2.5 | 4.75 | 1.65 | 11.7 | 1.2 |
| Peptide #4 | | | | | |
| Lipid with | 1.6 | 1.28 | 2.5 | 12 | 2 |
| Peptide #5 | | | | | |
| Lipid with | 1.13 | 1.75 | 1.93 | 34 | 4.2 |
| Peptide #6 | | | | | |
| Lipid with | 0.91 | 1.47 | 1.46 | 29 | 3.3 |
| Peptide #7 | | | | | |
| Lipid with | 0.37 | 1 | 1.86 | 19.9 | 1.6 |
| Peptide #8 | | | | | |
| Lipid with | 2.68 | 5.78 | 2.17 | 21.9 | 4.25 |
| Peptide #9 | | | | | |
| Lipid with | 1.4 | 2 | 2.1 | 22.7 | 2.5 |
| Peptide #10 | | | | | |

Peptide #1: (K)₁₆YYCDIRLRDP
Peptide #2: (K)₁₆NSRDSSGIQNV
Peptide #3: (K)₁₆AAWDDSLGI
Peptide #4: (K)₁₆VELDSFDY
Peptide #5: (K)₁₆DYYCAAWDDSLNGYSVF
Peptide #6: (K)₁₆YYCQQRSSYPYTFGG
Peptide #7: (K)₁₆YYCLQSMEDPYTFGG
Peptide #8: (K)₁₆YYCARSDGNYGYYYALDYDY
Peptide #9: (K)₁₆AARSPSYYRYDYGPYYAMDYD
Peptide #10: (K)₁₆YYCQQSYSTPWTFGQGTK.

These results demonstrate that significant and surprising enhancements of transfection efficiency can be achieved by using CDR peptides together with cationic lipid for transfection, as compared with cationic lipid alone.

1-37. (canceled)

38. A complex comprising:
a) a nucleic acid;
b) a transfection agent; and
c) a peptide derived from a CDR of a specific antibody, wherein said peptide comprises a sequence comprising at least 6 contiguous amino acids from a CDR2 or CDR3 sequence of said antibody, wherein said peptide is functionally linked to a polycationic moiety, and wherein said peptide binds to the same target as said antibody.

39. The complex according to claim 38, wherein said polycationic moiety comprises a sequence of amino acids, wherein said polycationic sequence has a charge of at least +8.

40. The complex according to claim 38, wherein said polycationic sequence comprises at least 8 lysine, arginine, or ornithine residues, and wherein said polycationic sequence is a polylysine, polyornithine, or polyarginine sequence, or a mixture of lysine, ornithine and/or arginine.

41. The complex according to claim 38, wherein said peptide further comprises a nuclear localization sequence.

42. The complex according to claim 38, wherein said polycationic moiety is covalently linked to said peptide.

43. The complex according to claim 42, wherein said polycationic moiety is selected from the group consisting of a polycationic peptide sequence, a polyamine, a peptide nucleic acid, spermine, spermidine and carboxyspeimidine.

44. The complex according to 38, wherein said transfection agent is a cationic lipid, and wherein said cationic lipid comprises at least one polyvalent cationic lipid.

45. The complex according to 38, wherein said complex further comprises a transfection enhancing agent, wherein the transfection enhancing agent comprises a nuclear localization protein or peptide.

46. The complex according to claim 38, wherein said nucleic acid is a vector or an expression vector, wherein said expression vector encodes a protein.

47. The complex according to claim 46, wherein said nucleic acid is an expression vector and said protein is an antibody or a reporter protein.

48. A method for introducing a nucleic acid, protein, or peptide into a cell, comprising contacting the cell with a complex according to claim 38.

49. The method according to claim 48, wherein said polycationic moiety is covalently linked to said peptide.

50. The method according to claim 48, wherein said cell is a suspension cell.

51. A complex comprising:
a) a nucleic acid;
b) a transfection agent; and
c) a peptide derived from a CDR of a specific antibody, wherein said peptide comprises a modified amino acid sequence having at least 80% or at least 90% sequence identity to an unmodified sequence of at least 7 contiguous amino acids from a CDR2 or CDR3 sequence of said antibody, wherein said peptide is functionally linked to a polycationic moiety, and wherein said modified peptide retains the ability to bind the target recognized by said unmodified peptide.

52. The complex according to claim 51, wherein said peptide comprises an amino acid sequence having at least 80% or at least 90% sequence identity to a sequence selected from the group consisting of: YYCDIRLRDP, NSRDSSGIQNV, AAWDDSLGI, VELDSFDY, DYYCAAWDDSLNGYSVF, YYCQQRSSYPYTFGG, YYCLQSMEDPYTFGG, YYCARSDGNYGYYYALDYDY, AARSPSYYRYDYGPYYAMDYD, and YYCQQSYSTPWTFGQGTK.

53. A method for introducing a nucleic acid, protein, or peptide into a cell, comprising contacting the cell with a complex according to claim 51.

54. A kit comprising a transfection agent, and a peptide derived from a CDR of a specific antibody, wherein said peptide comprises an amino acid sequence having at least 80% or at least 90% sequence identity to a sequence of at least 7 contiguous amino acids from a CDR2 or CDR3 sequence of said antibody and wherein said peptide is functionally linked to a polycationic moiety.

55. The kit according to claim 54, wherein said transfection agent is a cationic lipid transfection agent.

56. A kit according to claim 54 further comprising a peptide comprising a nuclear localization sequence.
